(19) United States
(12) Patent Application Publication
(10) Pub. No.: US 2021/0177531 A1
(43) Pub. Date: Jun. 17, 2021

(54) ROBOTIC SURGICAL SYSTEM AND METHODS OF USE THEREOF

(71) Applicant: Covidien LP, Mansfield, MA (US)

(72) Inventors: Amanda H. Lennartz, Erie, CO (US); Kenlyn S. Bonn, Lakewood, CO (US); Tyler J. Bagrosky, Arvada, CO (US)

(21) Appl. No.: 17/101,616

(22) Filed: Nov. 23, 2020

Related U.S. Application Data
(60) Provisional application No. 62/946,507, filed on Dec. 11, 2019.

(51) Int. Cl. A61B 34/30; A61B 5/02; A61B 5/026; A61B 90/00

(52) U.S. Cl. CPC A61B 34/30; A61B 5/02042; A61B 2034/105; A61B 90/37; A61B 2034/302; A61B 5/0261

(57) ABSTRACT

A method of performing a minimally-invasive surgical procedure includes: determining that a blood vessel within tissue has a hemorrhage; determining a location of the hemorrhage; and displaying over a digital image of the blood vessel the determined location of the hemorrhage.

### CROSS-REFERENCE TO RELATED APPLICATION

This application claims the benefit of and priority to U.S. Provisional Patent Application No. 62/946,507 filed Dec. 11, 2019, the entire disclosure of which is incorporated by reference herein.

### INTRODUCTION

The present disclosure relates to methods of performing surgical procedures. More particularly, the present disclosure relates to methods and apparatus for performing minimally-invasive robotic surgical procedures.

### BACKGROUND

Surgical techniques and instruments have been developed that allow a surgeon to perform an increasing range of surgical procedures with minimal incisions into the skin and body tissue of the patient. Minimally-invasive surgery has become widely accepted in many medical specialties, often replacing traditional open surgery. Unlike open surgery, which requires a long incision, minimally-invasive procedures, such as endoscopy or laparoscopy, are performed through one or more short incisions, with much less trauma to the body.

In laparoscopic and endoscopic surgical procedures, a small “keyhole” incision or puncture is made in a patient's body, e.g., in the abdomen, to provide an entry point for a surgical access device which is inserted into the incision and facilitates the insertion of specialized instruments used in performing surgical procedures within an internal surgical site. The number of incisions may depend on the type of surgery. It is not uncommon for some abdominal operations, e.g., gallbladder surgery, to be performed through a single incision. In most patients, the minimally-invasive approach leads to decreased postoperative pain, shorter hospital stay, faster recovery, decreased incidence of wound-related and pulmonary complications, cost savings by reducing post-operative care, and, in some cases, a better overall outcome.

In minimally-invasive surgery, the surgeon does not have direct visualization of the surgical field, and thus minimally-invasive techniques require specialized skills compared to the corresponding open surgical techniques. Although minimally-invasive techniques vary widely, surgeons generally rely on a lighted camera at the tip of an endoscope to view the surgical site, with a monitor displaying a magnified version of the site for the surgeon to use as a reference during the surgical procedure. The surgeon then performs the surgery while visualizing the procedure on the monitor.

### SUMMARY

In one aspect of the present disclosure, a method of performing a minimally-invasive robotic surgical procedure is provided and includes: inserting a surgical instrument into a surgical site; treating tissue in the surgical site with the surgical instrument; determining, using a sensor, that a blood vessel within the tissue has a hemorrhage; and sealing the blood vessel after determining that the blood vessel has the hemorrhage.

In some aspects, the method may further include moving blood away from the blood vessel after determining that the blood vessel has the hemorrhage.

In some aspects, the method may further include locating the hemorrhage after the blood is moved away.

In some aspects, determining that the blood vessel has the hemorrhage may include measuring local perfusion in a plurality of locations of the tissue.

In some aspects, the blood vessel may be determined to have the hemorrhage when the local perfusion is higher in the blood vessel compared to surrounding tissue.

In some aspects, the method may further include generating a digital image of vasculature in the tissue; and displaying an image of the tissue and the digital image of the vasculature on a display. The digital image of the vasculature may overlay the image of the tissue.

In some aspects, the method may further include determining a location of the hemorrhage and displaying over the digital image of the vasculature the determined location of the hemorrhage.

In some aspects, the method may further include displaying the surgical instrument on the display and guiding the surgical instrument to the location of the hemorrhage.

In some aspects, the method may further include displaying the digital image of the vasculature on the display in a color different than an actual color of the vasculature.

In some aspects, the method may further include changing the color of the digital image of the vasculature based on a temperature of the vasculature.

In some aspects, the sensor may be a Doppler flow sensor that measures local perfusion through each of a plurality of sections of the tissue.

In some aspects, the method may further include displaying an image of the tissue on a display and overlaying on the displayed image of the tissue a representation of the measured local perfusion of each of the plurality of sections of the tissue.

In some aspects, the method may further include locating the hemorrhage based on the displayed representation of the measured local perfusion of each of the plurality of sections of the tissue.

In some aspects, the method may further include generating an infrared image of the tissue and displaying the infrared image of the tissue on a display.

In some aspects, the method may further include identifying a cauterized portion of the tissue by viewing the displayed infrared image of the tissue.

In accordance with another aspect of the present disclosure a method of performing a minimally-invasive robotic surgical procedure is provided and includes: displaying an image of tissue on a display; displaying a digital image of vasculature of the tissue overlaid on the displayed image of the tissue; determining that a blood vessel within the tissue has a hemorrhage; determining a location of the hemorrhage; and displaying over the digital image of the vasculature the determined location of the hemorrhage.

In some aspects, the method may further include sealing the blood vessel with a surgical instrument at the hemorrhage.

In some aspects, the method may further include displaying the surgical instrument on the display and guiding the surgical instrument to the location of the hemorrhage using the displayed surgical instrument and the displayed location of the hemorrhage.

In some aspects, determining that the blood vessel has the hemorrhage may include measuring local perfusion in a plurality of locations of the tissue.

In some aspects, the blood vessel may be determined to have the hemorrhage when the local perfusion is higher in the blood vessel compared to surrounding tissue.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the present disclosure are described herein with reference to the accompanying drawings, wherein:

FIG. 1 is a schematic diagram of a robotic surgical system provided in accordance with aspects of the present disclosure;

FIG. 2A is a front view of a display of the robotic surgical system of FIG. 1 illustrating an actual image of tissue and vasculature thereof at a surgical site;

FIG. 2B is a front view of the display of FIG. 2A illustrating the actual image of the tissue and the vasculature thereof with a digital image of the vasculature superimposed thereon, the digital image of the vasculature identifying a location of a hemorrhage; and

FIG. 3 is a flowchart illustrating an exemplary method for performing a surgical procedure utilizing the robotic surgical system of FIG. 1.

### DETAILED DESCRIPTION

Embodiments of the disclosed robotic surgical system and methods of use thereof are described in detail with reference to the drawings, in which like reference numerals designate identical or corresponding elements in each of the several views. As used herein the term “distal” refers to that portion of the robotic surgical system or component thereof, that is closer to a patient, while the term “proximal” refers to that portion of the robotic surgical or component thereof, that is farther from the patient.

This disclosure relates to a robotic surgical system including a camera for capturing images of tissue in a surgical site and infrared light transmitters and sensors for detecting and imaging vasculature disposed underneath the surface of the tissue. A processor generates a digital image of the vasculature (e.g., veins) using data acquired by the infrared sensors. The processor is in communication with a display configured to display an actual image of the tissue and vasculature captured by the camera. The processor superimposes the digital image of the vasculature over the actual image of the vasculature to provide a clinician with a clear view of where the vasculature is located relative to the outer surface of the tissue.

Due to the difficulty in visually identifying the presence of a hemorrhage in a surgical site, which may be obscured by blood, the robotic surgical system is configured to identify the hemorrhage and display the location of the hemorrhage on the digital image of the vasculature. The robotic surgical system, or a clinician, may use the identified location of the hemorrhage to repair the hemorrhage using a suitable surgical instrument operatively coupled to the robotic surgical system. Overlaying the blood vessel may help prevent bleeding due to surgeon manipulation.

The robotic surgical system of this disclosure utilizes near-infrared (NIR) light to image or visualize to various depths within tissue. Veins contain de-oxygenated hemoglobin, which has a near infrared absorption peak at about 760 nm and a lesser, more broad absorption plateau over the range of 800 nm to 950 nm. There is a window of wavelengths in the near infrared region between 650 nm and 900 nm in which photons are able to penetrate tissue far enough to illuminate deeper structures beyond depths of 1 cm. The robotic surgical system of this disclosure takes advantage of this phenomenon by using near-infrared wavelengths of approximately 880 nm to 890 nm for imaging subcutaneous veins in tissue.

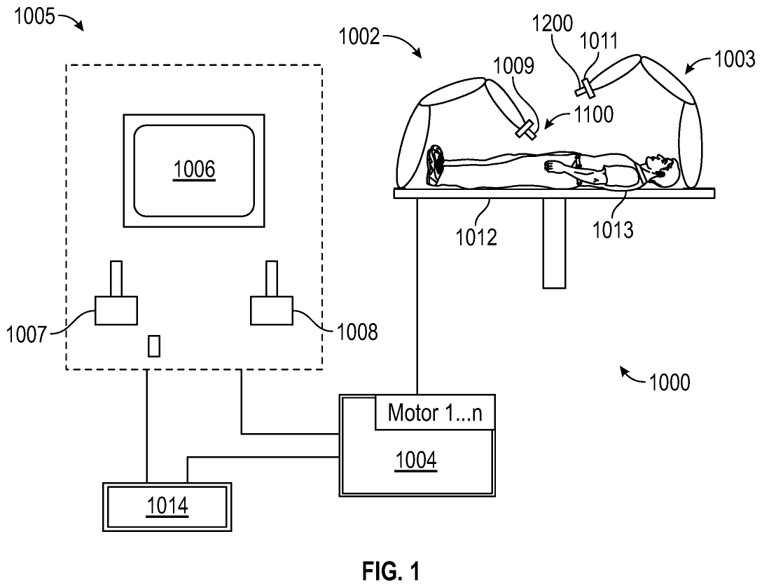

With reference to FIG. 1, a robotic surgical system exemplifying the aspects and features of the present disclosure is shown identified by reference numeral 1000. Robotic surgical system 1000 includes a plurality of robot arms 1002, 1003; a control device 1004; and an operating console 1005 coupled with control device 1004. Operating console 1005 may include a display 1006, which may be set up in particular to display three-dimensional images; and manual input devices 1007, 1008, to enable a surgeon to telemanipulate robot arms 1002, 1003. Robotic surgical system 1000 may be configured for use on a patient 1013 lying on a patient table 1012 to be treated in a minimally invasive manner. Robotic surgical system 1000 may further include a database 1014 coupled to control device 1004, in which pre-operative data from patient 1013 and/or anatomical atlases are stored. Each of the robot arms 1002, 1003 may include a plurality of segments, which are connected through joints, and an attaching device 1009, 1011, to which may be attached, for example, an end effector assembly 1100, 1200, respectively.

Robot arms 1002, 1003 and the end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

The control device 1004 may include a processor (not shown) connected to a computer-readable storage medium or a memory, which may be a volatile type memory, such as RAM, or a non-volatile type memory, such as flash media, disk media, or other types of memory. In various embodiments, the processor may be another type of processor such as, without limitation, a digital signal processor, a microprocessor, an ASIC, a graphics processing unit (GPU), field-programmable gate array (FPGA), or a central processing unit (CPU). In various embodiments, the memory can be random access memory, read-only memory, magnetic disk memory, solid-state memory, optical disc memory, and/or another type of memory. The memory may communicate with the processor through communication buses of a circuit board and/or through communication cables such as serial ATA cables or other types of cables. The memory includes computer-readable instructions that are executable by the processor to operate the end effector assembly 1200.

Manual input devices 1007, 1008 of robotic surgical system 1000 may further include a motion activation control, a motion-sensing assembly including a motor, rotation and/or articulation lockout features, excessive torque limiting features, and/or a rotation control, similarly as detailed above, to provide the user with the ability to control manipulation of end effector assemblies 1100, 1200, by moving manual input devices 1007, 1008 relative to a reference position.

The end effector assembly 1100 or 1200 may be any suitable surgical instrument suitable for use with the robotic surgical system 1000 including, but not limited to, a bipolar instrument, a monopolar instrument, an ablation instrument, a thermal treatment instrument, an ultrasonic instrument, a tissue grasper, a surgical stapler, a microwave instrument, or a radiofrequency instrument. It is contemplated that the robotic surgical system 1000 may include a surgical instrument separate from the robot arm 1002, 1003 for manual control by a clinician.

The end effector assembly 1100 or 1200 includes one or more perfusion sensors, for example, a Doppler flow sensor, configured to measure local perfusion (e.g., blood flow) through tissue. In some aspects, a hand-held, laparoscopic surgical instrument may be provided having one or more perfusion sensors attached to a distal end thereof. The perfusion sensors may measure perfusion of tissue on the basis of known techniques, such as Laser-Doppler Flowmetry (“LDF”), measuring light scattering, and/or measuring absorption of light from one or more LED's or other light sources.

The perfusion sensors are in communication, via lead wires or wireless connection, with the display 1006 such that upon the sensors measuring perfusion in tissue, the sensors transmit the measurement data to the display 1006, which displays the measurement using a number, word, or image. In some embodiments, the sensors may also be in communication, via lead wires or wireless connection, with a computing device or processor (not shown) such as a laser Doppler monitor, which processes the information collected by the sensors to calculate the tissue perfusion. The computing device (e.g., a laser Doppler monitor) may also be in communication, via lead wires or wireless connection, with the display 1006 to send the processed information related to the tissue perfusion to the display 1006 so that the display 1006 can display the local tissue perfusion measurements.

One or more of the end effector assemblies 1100 or 1200 may include an infrared transmitter, such as, for example, infrared light-emitting diodes (“IR-LEDs”) or lasers for transmitting near-infrared light, and one or more infrared receivers or sensors for receiving near-infrared light. In some aspects, a hand-held, laparoscopic surgical instrument may be provided having the infrared transmitter the infrared receiver attached to a distal end thereof. The infrared receivers may be an infrared sensitive optical sensor such as, for example, a charge coupled device (“CCD”) sensor array, a complementary metal oxide semiconductor (“CMOS”) sensor array, a phototransistor sensor array or the like. In embodiments, the infrared transmitters and infrared receivers may be configured as one sensor having both infrared transmission and reception capability.

The infrared transmitters and receivers are in communication with the processor of the control device 1004 for generating a digital image of vasculature targeted by the infrared transmitters. The processor is in communication with the infrared transmitters and receivers. As such, the amount of infrared light transmitted to tissue by the infrared transmitters and the amount of infrared light received by the infrared receivers is known by the processor. The processor is configured to use this data to generate a digital image of the vasculature targeted by the infrared transmitters.

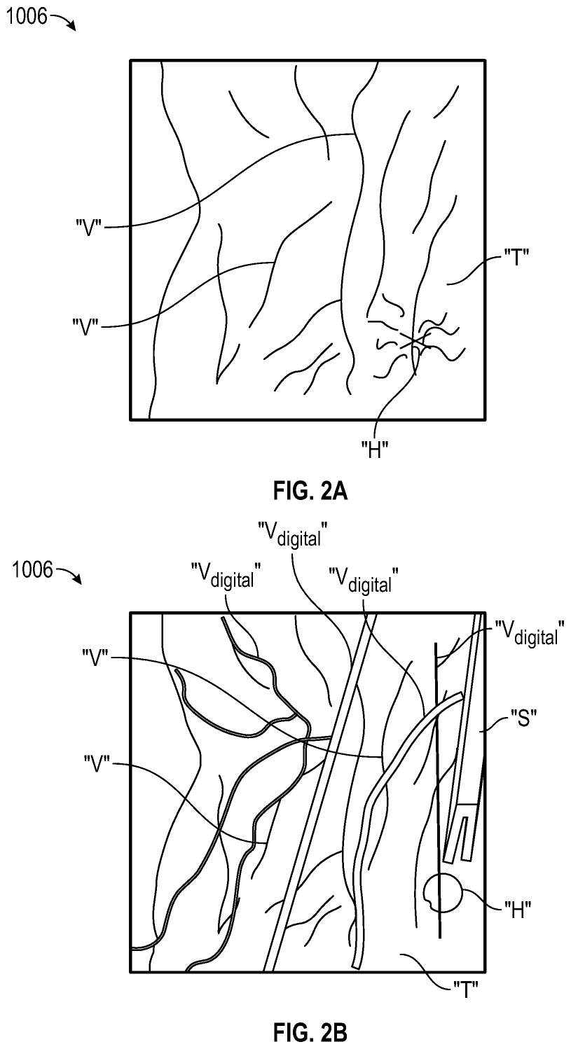

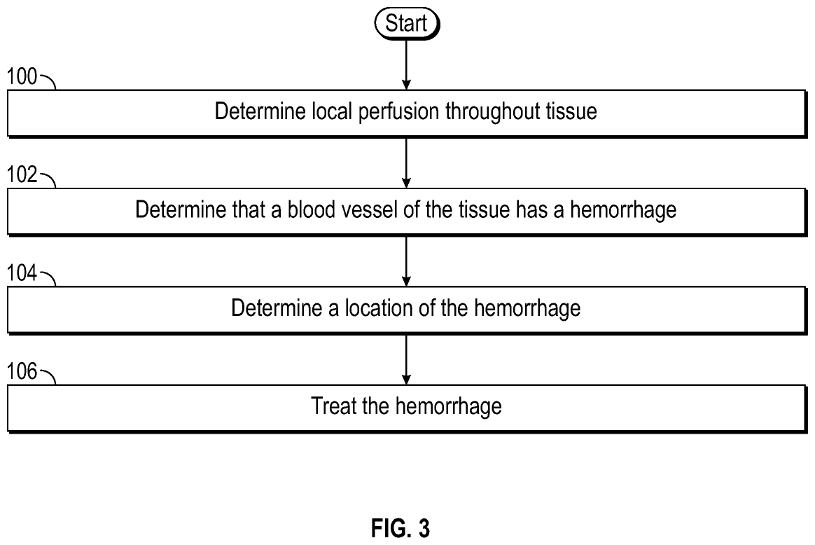

With reference to FIGS. 2A, 2B, and 3, a method of treating tissue utilizing the robotic surgical system 1000 of FIG. 1 will now be described. It is contemplated that the methods of treating tissue described herein may alternatively be performed by a clinician without the assistance of the robotic surgical system 1000.

In operation, a minimally invasive surgical procedure may require knowledge of the location of vasculature “V” underneath tissue “T” and/or any hemorrhages “H” that may result in the vasculature “V” to allow a clinician to rapidly identify and treat the hemorrhage “H.” To locate and view the vasculature “V,” an endoscope is passed through a port assembly to position a distal end portion of the endoscope adjacent the tissue “T.” The endoscope captures an image (e.g., video or a still image) of the tissue “T” and displays the image of the tissue “T” on the display 1006, as shown in FIG. 2A. Since vasculature disposed underneath tissue is typically at least partially visible, the image of the tissue on the display 1006 will also show the vasculature “V.”

Concurrently with capturing the image of the tissue “T” with the endoscope, the infrared transmitters of the endoscope transmit infrared light toward the tissue “T.” Due to the difference in infrared-absorption capability between tissue (e.g., muscle, skin, fat) and vasculature, most of the infrared light directed at the tissue “T” without vasculature “V” reflects back toward the endoscope, whereas most of the infrared light directed at the tissue “T” having the vasculature “V” disposed underneath gets absorbed by the vasculature “V.” The infrared light that gets reflected by the tissue “T” is received by the infrared receivers, which communicate the data to the control device 1004 (FIG. 1).

The control device 1004, using the data received from the infrared receivers, locates/identifies the vasculature “V” and generates a digital image of the vasculature “Vdigital” (FIG. 2B). The control device 1004 relays the digital image of the vasculature “Vdigital” to the display 1006, and the display 1006 superimposes the digital image of the vasculature “Vdigital” on the actual image of the vasculature “V” captured by the endoscope. The clinician, now with a better visualization of the vasculature “V,” may more effectively navigate around the vasculature “V” or treat the vasculature “V” depending on the surgical procedure being performed. The clinician may then treat the tissue “T” using end effector assembly 1100 or 1200. Treating the tissue “T” may include, for example, sealing and cutting the tissue “T” using a vessel sealer or sealing the tissue “T” by grasping the tissue “T” with a tissue grasper.

In embodiments, the digital image of the vasculature “Vdigital” displayed on the display 1006 may be a color different than the actual color of the vasculature “V.” For example, the digital image of the vasculature “Vdigital” may be displayed in yellow, green, blue, or any suitable color and may change based on a measured temperature of different portions of the tissue “T”.

Prior to, during, or after treating the tissue “T,” in step 100, the perfusion sensors of the end effector assembly 1200 are positioned over the tissue “T” and determine local tissue perfusion throughout a plurality of sections of the tissue “T” around the treatment site. A visual representation (e.g., a number, letter, or the like) of the measured local perfusion of each of the plurality of sections of the tissue “T” may be overlaid on the displayed image of the tissue “T” to assist a clinician in determining whether blood flow throughout the tissue “T” is normal.

During some surgical procedures, a hemorrhage in the treated tissue may occur without the knowledge of the clinician given that the presence of blood may not always be abnormal. In step 102, a blood vessel of the vasculature “V” is determined to have a hemorrhage “H” when the local perfusion in a specific location of the vasculature “V is higher compared to surrounding tissue. This may occur due to the hemorrhage “H” allowing blood to flow freely out of the opening in the blood vessel with little resistance. In step 104, the control device 1004 may determine the location of the hemorrhage “H” using the data from the perfusion sensors. It is contemplated that the presence and location of a hemorrhage may be determined using other suitable methods, such as a camera configured to distinguish between normal and abnormal blood flow.

In aspects, the presence and location of the hemorrhage may be determined using Acoustic Doppler Velocimetry. For example, acoustic Doppler velocimeter sensors may be attached to the distal end of the endoscope or a trocar that provides access into the surgical site for the endoscope. When a blood vessel is hemorrhaging, the sensors (e.g., three sensors) may generate a Doppler signature that represents the hemorrhaging blood vessel.

In step 106, upon locating the hemorrhage “H,” the hemorrhage “H” may be automatically sealed using a robotically-operated vessel sealer.

In some aspects, a clinician, instead of the robotic surgical system 1000, may control the vessel sealer to treat the hemorrhage “H.” In particular, upon the robotic surgical system 1000 identifying and locating the hemorrhage “H,” the robotic surgical system 1000 may display over the digital image of the vasculature “Vdigital” the determined location of the hemorrhage “H,” as shown in FIG. 2B. As shown in FIG. 2B, a surgical instrument “S” (e.g., vessel sealer, tissue grasper, or surgical stapler) may be displayed on the display 1006 allowing the clinician to guide the surgical instrument “S” to the location of the hemorrhage “H” using the displayed surgical instrument “S” and the displayed location of the hemorrhage “H.” Upon properly positioning the surgical instrument “S” relative to the hemorrhage “H,” the clinician may seal the blood vessel with the surgical instrument “S.” In some aspects, the robotic surgical system 1000 may use a surgical irrigator, vacuum, or the like to move blood away from the bleeding blood vessel, such that the clinician may locate the hemorrhage “H” and then treat the hemorrhage “H” without the assistance of the display 1006.

In embodiments, the robotic surgical system 1000 may include a temperature sensor (not shown) for determining a temperature of the tissue and/or vasculature “V.” The control device 1004 or the display 1006 may utilize the temperature of the vasculature “V” determined by the temperature sensor to generate an infrared image of the vasculature based on the temperature of the vasculature “V.” For example, if the temperature of the vasculature “V” is cooler than a known baseline temperature (e.g., 98.6° F.) or range of temperatures (e.g. 95° F.-99° F.), then the digital image of the vasculature “V” may be displayed as blue, whereas if the temperature of the vasculature “V” is warmer than the known baseline temperature or range of temperatures, then the digital image of the vasculature “V” may be displayed as yellow. Due to the differences in temperature of cauterized tissue versus healthy tissue, any inadvertent burns in the tissue or vasculature “V” thereof will be viewable on the displayed infrared image of the tissue.

The flow diagram described above includes various blocks described in an ordered sequence. However, those skilled in the art will appreciate that one or more blocks of the flow diagram may be performed in a different order, repeated, and/or omitted without departing from the scope of the disclosure. The above description of the flow diagram refers to various actions or tasks performed by the robotic surgical system 1000, but those skilled in the art will appreciate that the robotic surgical system 1000 is exemplary. In various embodiments, the disclosed operations can be performed by a clinician or another component, device, or system. In various embodiments, the robotic surgical system 1000 or other component/device performs the actions or tasks via one or more software applications executing on the processor. In various embodiments, at least some of the operations can be implemented by firmware, programmable logic devices, and/or hardware circuitry. Other implementations are contemplated to be within the scope of the disclosure.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A method of performing a minimally-invasive robotic surgical procedure, comprising:
inserting a surgical instrument into a surgical site;
treating tissue in the surgical site with the surgical instrument;
determining, using a sensor, that a blood vessel within the tissue has a hemorrhage; and
sealing the blood vessel after determining that the blood vessel has the hemorrhage.

2. The method according to claim 1, further comprising moving blood away from the blood vessel after determining that the blood vessel has the hemorrhage.

3. The method according to claim 2, further comprising locating the hemorrhage after the blood is moved away.

4. The method according to claim 1, wherein determining that the blood vessel has the hemorrhage includes measuring local perfusion in a plurality of locations of the tissue.

5. The method according to claim 4, wherein the blood vessel is determined to have the hemorrhage when the local perfusion is higher in the blood vessel compared to surrounding tissue.

6. The method according to claim 1, further comprising:
generating a digital image of vasculature in the tissue; and
displaying an image of the tissue and the digital image of the vasculature on a display, wherein the digital image of the vasculature overlays the image of the tissue.

7. The method according to claim 6, further comprising:
determining a location of the hemorrhage; and
displaying over the digital image of the vasculature the determined location of the hemorrhage.

8. The method according to claim 7, further comprising:
displaying the surgical instrument on the display; and
guiding the surgical instrument to the location of the hemorrhage.

9. The method according to claim 6, further comprising displaying the digital image of the vasculature on the display in a color different than an actual color of the vasculature.

10. The method according to claim 9, further comprising changing the color of the digital image of the vasculature based on a temperature of the vasculature.

11. The method according to claim 1, wherein the sensor is a Doppler flow sensor that measures local perfusion through each of a plurality of sections of the tissue.

12. The method according to claim 11, further comprising:
displaying an image of the tissue on a display; and
overlaying on the displayed image of the tissue a representation of the measured local perfusion of each of the plurality of sections of the tissue.

13. The method according to claim 12, further comprising locating the hemorrhage based on the displayed representation of the measured local perfusion of each of the plurality of sections of the tissue.

14. The method according to claim 1, further comprising:
generating an infrared image of the tissue; and
displaying the infrared image of the tissue on a display.

15. The method according to claim 14, further comprising identifying a cauterized portion of the tissue by viewing the displayed infrared image of the tissue.

16. A method of performing a minimally-invasive robotic surgical procedure, comprising:
displaying an image of tissue on a display;
displaying a digital image of vasculature of the tissue overlaid on the displayed image of the tissue;
determining that a blood vessel within the tissue has a hemorrhage;
determining a location of the hemorrhage; and
displaying over the digital image of the vasculature the determined location of the hemorrhage.

17. The method according to claim 16, further comprising sealing the blood vessel with a surgical instrument at the hemorrhage.

18. The method according to claim 17, further comprising:
displaying the surgical instrument on the display; and
guiding the surgical instrument to the location of the hemorrhage using the displayed surgical instrument and the displayed location of the hemorrhage.

19. The method according to claim 16, wherein determining that the blood vessel has the hemorrhage includes measuring local perfusion in a plurality of locations of the tissue.

20. The method according to claim 19, wherein the blood vessel is determined to have the hemorrhage when the local perfusion is higher in the blood vessel compared to surrounding tissue.
